(19) United States
(12) Patent Application Publication
(10) Pub. No.: US 2018/0228426 A1
(43) Pub. Date: Aug. 16, 2018

(54) Image Processing System and Method

(71) Applicant: EMERALD MEDICAL APPLICATION LTD., Modiin (IL)

(72) Inventors: Ilan SINAI, Pardes Hanna (IL); Marina ASHEROV, Rishon Lezion (IL); Lior WAYN, Modiin (IL); Adi ZAMIR, Kfar Saba (IL)

(73) Assignee: Emerald Medical Applications Ltd., Modiin (IL)

(21) Appl. No.: 15/748,884

(22) PCT Filed: Jul. 28, 2016
(86) PCT No.: PCT/IL2016/050828

§ 371 Date: Jan. 30, 2018

(30) Foreign Application Priority Data
Jul. 30, 2015 (GB) 1513455.4

(51) Int. Cl. A61B 5/00; G06K 9/46; G06K 9/34; G06T 7/11; G06K 9/62; G06T 7/194; G06T 7/90

(52) U.S. Cl. CPC A61B 5/444; G06K 9/4652; G06K 9/34; G06T 7/11; G06T 2207/30088; G06T 7/194; G06K 9/629; G06T 7/90; G06T 2207/10024; G06K 9/6267

(57) ABSTRACT

A computerized system and method for analyzing a digital photograph and identifying skin areas, the method comprises three main steps. At first, transforming the digital photograph to a plurality of color bases candidates, and choosing one candidate based on highest score plane. Then, performing a segmentation of the digital photograph to a plurality of objects based on maximal separations between different object. Lastly, classifying the objects to one or more of the following categories: skin, background or underwear.

### TECHNICAL FIELD

The present invention relates to image processing in general, and in particular to analyzing digital photographs of people and identifying skin areas.

### BACKGROUND ART

Skin lesions can be a source of concern because a normal person may not know where they are and may not be conscious of or follow bow their shape, size, and color are and if they have changed recently. Knowing this information may direct the person on: which lesions should be treated, and most of all alert the person to contact a physician as early as possible, thus giving the physician/person precious time to be able to decide whether these lesions are malignant (cancerous) or benign.

It has become more and more popular to use technology as a source of information and assistance. Since many people today have mobile phones equipped with a camera, it is possible for a person to photograph areas of his body surrounding the skin lesion, so the photograph can be analyzed automatically by an application.

Before an application can analyze a skin lesion, there is a need to process the digital photograph and identity only the bare skin areas, and disregard background areas and areas with clothing or jewelry obstructing the skin view.

### SUMMARY OF INVENTION

It is an object of the present invention to provide a system for analyzing a digital photograph.

It is an object of the present invention to provide a system for analyzing a digital photograph and identifying skin areas.

It is a further object of the present invention to provide a system for analyzing a digital photograph and identifying background area.

It is yet another object of the present invention to provide a system for analyzing a digital photograph and identifying clothing, jewelry and other wearable objects.

It is yet a further object of the present invention to provide a system for analyzing a digital photograph and identifying hair areas.

It is yet another object of the present invention to provide a system for analyzing a digital photograph and identifying whether the photograph was taken in portrait or landscape views.

The present invention relates to a computing system comprising at least one processor; and at least one memory communicatively coupled to the at least one processor comprising computer-readable instructions that when executed by the at least one processor cause the computing system to implement a method for analyzing a digital photograph and identifying skin areas, the method comprising the steps of:
(i) transforming said digital photograph to a plurality of color bases candidates, and choosing one candidate based on highest score plane;
(ii) performing a segmentation of said digital photograph to a plurality of objects based on maximal separations between different object; and
(iii) classifying said objects to one or more of the following categories: skin, background or underwear, wherein said classification is based on chosen plane calculation, comprising the steps of:
a) using one or more selected label regions, computing a statistical decisor for each area;
b) sampling each of said regions one of more times;
c) calculating for each one-label region the statistical decisor;
d) calculating the statistical decisor for one of more features of every label; and
e) clarifying one-label regions with matching statistical decisor value as skin or assigning each object with a foreground probability and a background probability.

The matching statistical decisor can be, for example, the highest value, the lowest value etc. and is determined by the statistical calculation applied.

In some embodiments, the color base is hue-saturation-lightness (HSL), hue-saturation-value (HSV), hue-saturation-intensity (HSI) or La*b* (CIELa*b*) or RGB.

In some embodiments, the segmentation is based on hue or saturation or lightness or any one of the “regular” R or G or B planes.

In some embodiments, the segmentation comprises the following steps:
i) detecting thresholds using density estimation and maximization of inter class variation; and
(ii) smoothing and unifying objects in said digital photographs based or filters and proximity.

In some embodiments, the statistical decisor comprises average, common, median, mode, variance, standard deviation, (minimum+maximum)/2, any mathematical moment or any combination thereof.

In some embodiments, the feature comprises gray intensity, red/green/blue values, saturation value or any combination thereof.

In some embodiments, steps (a) to (e) of the classification are substituted by computing the statistical decisor for the entire object.

In some embodiments, the classifying, is based on object size count where either the biggest pixel count is classified as a foreground object or assigning each object with a foreground probability and a background probability.

In some embodiments, the classifying is based on central region counting, comprising the steps of:
(i) counting the number of pixels per object along a plurality of central lines;
(ii) classifying biggest pixel count objects as foreground objects or assign foreground and background probabilities per object;
(iii) counting the number of objects transitions along a plurality of central lines; and
(iv) classifying smallest pixel count objects as underwear objects or assign foreground and underwear probabilities per object.

In some embodiments, the classifying is based on peripheral region counting, comprising the steps of:
(i) counting the number of pixels per object along a plurality of peripheral left and right side lines; and
(ii) classifying biggest pixel count objects as background objects or assign foreground and background probabilities per object.

In some embodiments, the classifying is based on objects color and proximity to skin color, comprising the steps of:
(i) defining a common color per object; and
(ii) classifying closest color objects as foreground objects or assign foreground and underwear probabilities per object.

In some embodiments, the classifying is based on a scoring mechanism.

In some embodiments, the scoring mechanism is based on assigning each feature of an object a contributing weight factor that is multiplied by the calculated feature score, wherein the pixels labels with the biggest mark are classified as foreground objects, objects with smallest marks are classified as background objects, and objects with the highest object transitions count are classified as underwear objects.

In some embodiments, the computing system further comprises executing the step of determining image orientation.

In some embodiments, the image orientation is detected according to the following steps:
(i) detecting central lines as lines traversing from up to down and from left to right passing through the center of image or foreground center of mass;
(ii) counting the number of foreground pixels along a plurality of central lines;
(iii) counting the number of foreground and background pixels transitions along a plurality of central lines; and
(iv) determining the image as in portrait mode if count and transitions are higher and determining the image as in landscape mode if count and transitions are smaller.

In some embodiments, steps (i)-(iii) are repeated with off-central lines and adding their scores to the mechanism of step (iv).

In some embodiments, the computing system further comprises executing the step of head hair detection.

In some embodiments, the head hair detection is performed according to the following steps:
(i) scanning foreground pixels along top half central part;
(ii) counting 3D full color space density values of said foreground pixels;
(iii) clustering said values to a plurality of groups;
(iv) determining how close each cluster is to hair color;
(v) applying one or more edge filters on saturation or other color fields;
(vi) calculating variance of edge filter;
(vii) determining based on steps (i) to (vi) if a given, cluster is hair or not, wherein if a cluster is classified as hair, approximating head shape as geometric circle/oval shape using a voting method and classifying said cluster as non-bald head part and excluding said cluster from foreground/skin classification.

In some embodiments, the digital photograph is taken according to a total body photography (TBP) protocol. TBP or Whole Body Integumentary Photography is a well-established procedure for taking set of images that cover almost the entire body. These pictures are taken according to predefined set of body poses. The actual number of images taken can vary a little but it is usually around 25 pictures per person (range can be from 15 to 35 pictures per person). These sets include pictures taken from different angles, that is Front/Back/Left/Right side, covering the body from top to bottom. Additional images include feet, upper scalp and more.

In some embodiments, the label regions are selected randomly.

In another aspect, the present invention further relates to computerized system comprising a processor and a memory communicatively coupled to the processor comprising computer-readable instructions for analyzing a digital photograph and identifying skin areas, the system comprising:
(i) a software transformation module adapted for transforming said digital photograph to a plurality of color bases candidates, and choosing one candidate based on highest score plane;
(ii) a software segmentation module adapted for performing a segmentation of said digital photograph to a plurality of objects based on maximal separations between different object; and
(iii) a software classification module adapted for classifying said objects to one or more of the following categories: skin, background or underwear, wherein said classification is based on chosen plane calculation, comprising the steps of:
a) using one or more selected label regions, computing a statistical decisor for each area;
b) sampling each of said regions one or more times;
c) calculating for each one-label region the statistical decisor;
d) calculating the statistical decisor for one or more features of every label; and
e) classifying one-label regions with matching statistical decisor value as skin or assigning each object with a foreground probability and a background probability.

### BRIEF DESCRIPTION OF DRAWINGS

FIGS. 1A-1F show examples of 6 different density estimations using various planes. X ads shows increasing intensity values. Y axis show number of pixels.

FIGS. 2A-2F show six examples of thresholds outcomes based on different color planes. Axes are image coordinates.

FIGS. 3A-3B show an example of a segmented image (FIG. 3A) and improved images after cleanup (FIG. 3B). Axes are image coordinates.

FIGS. 4A-4B show a digital photograph of a torso (FIG. 4A) and an image where skin parts are identified in red (FIG. 4B). Axes are image coordinates.

FIGS. 5A-5B show randomly selected rectangle areas from different areas of the image. These rectangle are used in order to assign a tagging (skin/non skin) for the different objects/classes.

FIGS. 6A-6B show decision/outcome of such a tagging process. Detected skin parts are marked in deep blue. Axes are image coordinates.

FIG. 7 shows counts and transitions counts along several central and off central (peripheral). Lines can be seen as black lines. Axes are image coordinates.

FIG. 8 shows divisions to objects and background classes marked by black lines. The skin color reference is marked in star. X axis shows increasing intensity values. Y axis show number of pixels.

FIGS. 9A-9B show counts and transitions counts along several central and off central (peripheral) in order to detect image orientation. Lines can be seen as black lines. Axes are image coordinates.

FIG. 10 shows outcomes of hair patches filter. Axes are image coordinates.

FIG. 11 is a shape for explain morphological operations.

### MODES FOR CARRYING OUT THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

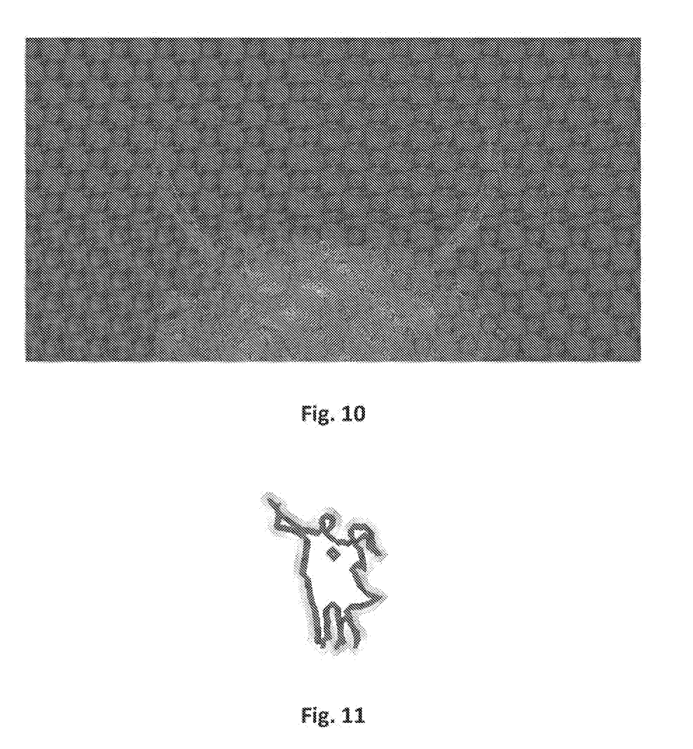

### GLOSSARY

| Term | Definition |
|---|---|
| Object/class/tag/ | Set of pixels, in a digital image, belonging to same |
| category | entity. Theses pixels were grouped together as the |
| | result of the segmentation and classification processes. |
| Tagging process | One of the objects of the invention. Each pixel in an |
| | analyzed image gets a: |
| | 1. Intermediate number called “label”, as the outcome |
| | of the segmentation process. |
| | 2. A tag, following all the classification steps. Possible |
| | tags/categories/classes (in the invention) are of the 3 |
| | categories below: |
| | 1. Foreground(Skin); |
| | 2. Background; |
| | 3. Underwear/tattoos/jewelry (Other). |
| Segmentation | In computer vision, image segmentation is the process |
| | of partitioning a digital image into multiple segments |
| | (sets of pixels). The goal of segmentation is to |
| | simplify and/or change the representation of an image |
| | into something that is more meaningful and easier to |
| | analyze. Image segmentation is typically used to locate |
| | objects and boundaries (lines, curves, etc.) in images. |
| | More precisely, image segmentation is the process of |
| | assigning a label to every pixel in an image such that |
| | pixels with the same label share certain characteristics. |
| Classification | In machine learning and statistics, classification is the |
| | problem of identifying to which of a set |
| | of categories (sub-populations) an object belongs. An |
| | example would be assigning a given email into “spam” |
| | or “non-spam” classes or assigning a diagnosis to a |
| | given patient as described by observed characteristics |
| | of the patient (gender, blood pressure, presence or |
| | absence of certain symptoms, ect.). |
| | In the invention, the objective is to identify and decide |
| | which label (found in the segmentation process) |
| | should be tagged as Skin, Background or Other. |
| Feature | A Feature is an individual measurable property of a |
| | phenomenon being observed or calculated. |
| Hard decision | For each class: Some of the features contribute to the |
| | final classification decision and some do not. |
| Soft Decision | For each class: All the features contribute to the final |
| | classification decision. |
| Morphological | FIG. 11 is shape (in blue) and its morphological |
| operations: | dilation (in green) and erosion (in yellow) by a |
| dilation, erosion. | diamond-shape structuring element |
| Morphological | These operations are based on dilation, erosion and |
| operations; | perform: |
| cleaning, | Cleaning—removing small isolated points (“debris”) |
| smoothing, | Smoothing—smooth sharp blob tips. |
| unifying | Unifying—merge several points that are nearby. |

The present invention relates to computerized system and method comprising a processor and memory for analyzing a digital photograph and identifying skin areas. It is important to isolate skin areas of a person, so that other applications and/or a human medical expert can analyze the skin areas, identify lesions and suggest possible treatment or further examination steps when necessary.

The digital photograph can come from many sources. For example, taken by a user via a mobile phone camera and analyzed on the mobile phone itself. The image can be taken by a regular camera or a mobile phone camera and then transferred to a personal computer for processing. The image (digital photograph) can also be loaded to a server, from a personal computer or mobile device, and then analyzed on a server. The user would then get the response via one or more communication methods, such as via un application on his computer or mobile device, an email message, an instant message etc.

A digital photograph can initially be in many formats. The two most common image format families today, for Internet images, are raster and vector. Common raster formats include JPEG, GIF, TIFF, BMP, PNG, HDR raster formats etc. Common vector image formats include GCM, Gerber format, SVG etc. Other formats include compound formats (EPS, PDF, PostScript etc.) and Stereo formats (MPO, PNS, JPS, etc.)

The first step in analyzing a digital image is to take the digital photograph and transform it to a plurality of color bases candidates. One candidate is chosen based on highest score plane. The color base can be hue-saturation-lightness (HSL), hue-saturation-value (HSV), hue-saturation-intensity (HSI), Lab or any other color base models.

Next, it is necessary to perform a segmentation of the digital photograph. The segmentation identifies a plurality of objects based on maximal separations between different objects. For each and every density distribution, a segmentation algorithm is performed. This algorithm suggests one or more threshold values. Implementing these threshold values will result in several distinct classes, each class consisting of all pixels that reside in the selected ranges of values. Typically, for N thresholds there will be N+1 classes (excluding minimum and maximum values). A class is assigned to all possible values (many times between 0 and 1). The segmentation algorithm also suggests a segmentation marking score. The better the separation/segmentation the higher the segmentation marking score.

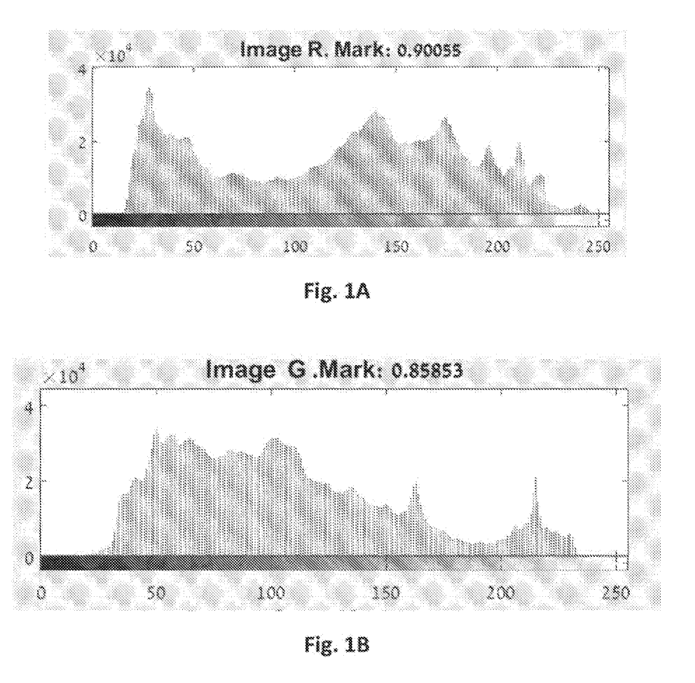

FIGS. 1A-1F show examples of graphs of density estimations using different planes. Each figure contains an interclass separation value, noted as Mark. FIGS. 1A-1C are density estimations of the Red (FIG. 1A), Green (FIG. 1B), and Blue (FIG. 1C) planes, all extracted from the raw RGB image.

FIG. 1D is a density estimation of the entire Red-Green-Blue channels treated together as grayscales (hence the name gray).

FIGS. 1E-1F are density estimations based on Hue/Saturation values calculated using the HSV transformation.

As can be seen, FIG. 1E has the highest interclass separation. This is reflected by its highest mark: 0.94707, therefore it will be chosen as the plane to process with.

The highest score plane is typically based oh Hue or color density though other parameters can also be used.

Segmentation involves three steps:

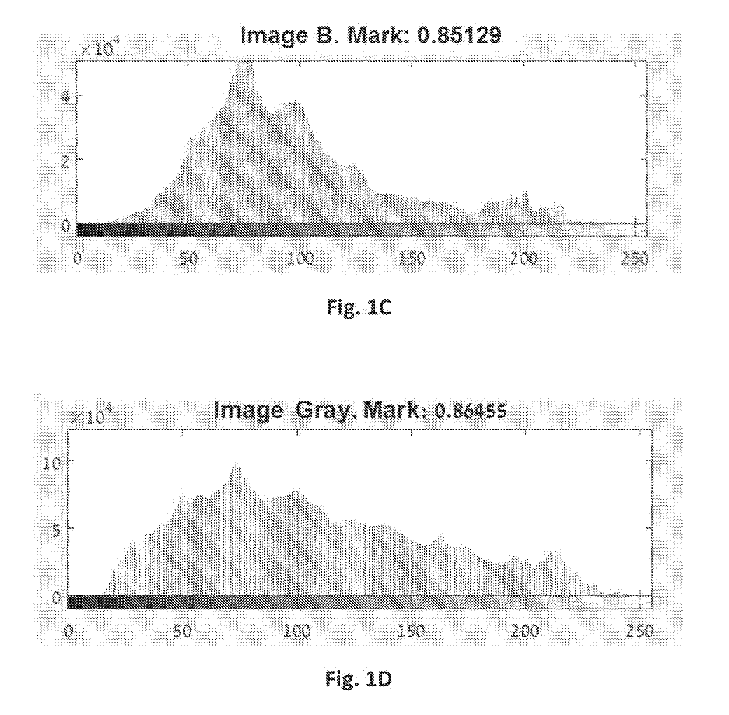

First, an automatic detection of threshold is performed using density estimation and maximization of the inter class color variance. FIGS. 2A-2F show six examples of thresholds outcomes based on different color planes and threshold performed on each plane. In FIGS. 1A-1E each segmentation for each plane was assigned a mark, where the image with the highest mark was the image with the best segmentation result (FIG. 1E). Looking at FIG. 2E one can verify that the best mark of 0.94707 indeed corresponds to the best segmentation among FIGS. 2A-2F. It is clear visually that FIG. 2E is indeed the best picture that separates skin, non-skin and background.

Second, the selected image needs to be further processed in order for the segmentation to be of good quality. The further processing comprises cleaning, smoothing and unifying objects in the digital photograph (image) based on filters. Filters size can be any number between 1-15 and it can be one dimension or two dimensions. The filters' coefficients can be any number between 0 to 1.

For example 6×4 filter can be [1, 1, 1, 1, 1, 1
1, 1, 0.5, 0.5, 1, 1
1, 1, 0.5, 0.5, 1, 1
1, 1, 1, 1, 1, 1].
   Or 9×1 filter of the form [1, 1, 0.75, 0.75, 0, 0.75, 0.75, 1, 1].

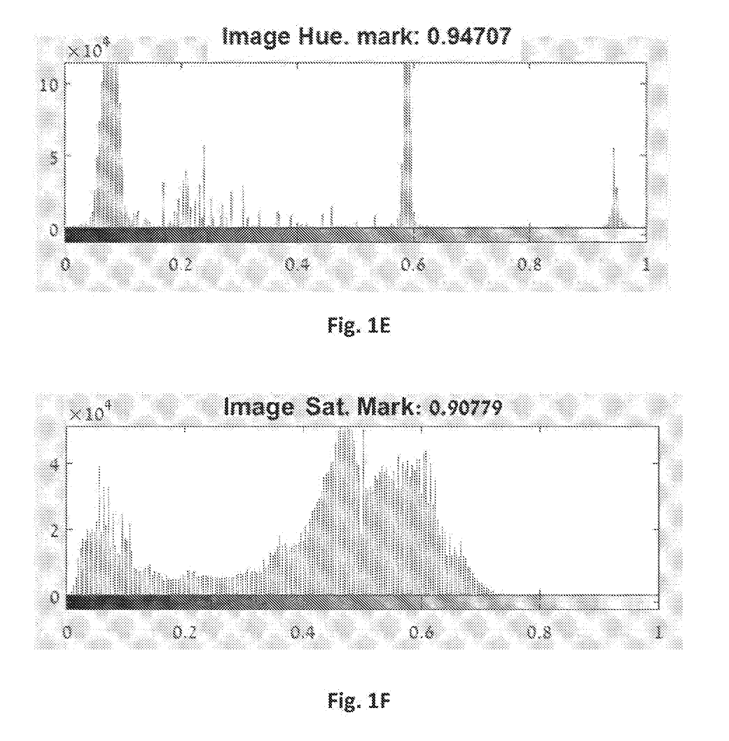

FIGS. 3A-3B show an example of a segmented image (FIG. 3A) which is eroded and dilate, and after cleaning, smoothing and being unified, the results are shown in FIG. 3B. As can be seen, the noise which is represented in yellow is reduced when moving from FIG. 3A to FIG. 3B.

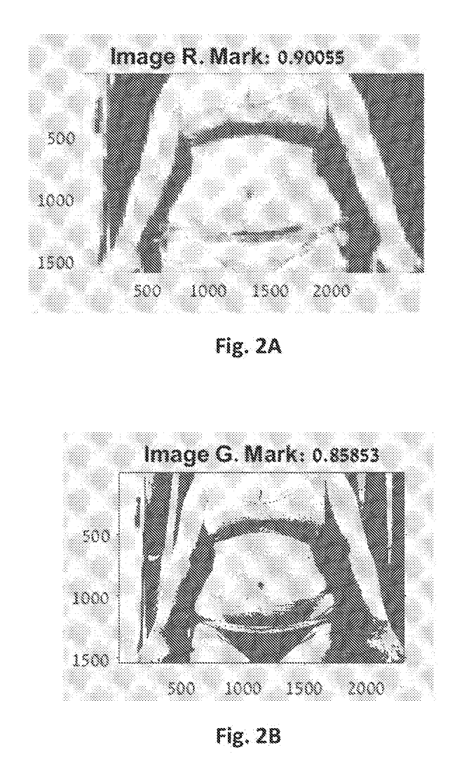

FIGS. 4A, 4B show a digital photograph of a torso (FIG. 4A) and an image where skin parts are identified in red (FIG. 4B). Some noise (yellow) is still present at the image of FIG. 4B. As can be seen most of the skin area was identified correctly and shown in red.

The final step of the segmentation process is labeling. Each identified pixel in the RGB image is assigned a label number. All pixels with the same label are declared as belonging to the same entity. Looking in FIG. 4B, each color represents a different label and is visualized by different color. The colors in FIG. 4B being red (for skin), blue (background) and yellow (non skin).

After different objects have identified in the digital photograph via the segmentation process, the next step is classifying these objects to one or more categories (also referred to as classes). Typically, the categories/classes/regions comprise: skin (foreground), background and underwear. In FIG. 4B, red represents skin, blue represents background and yellow represents non-skin or other (noise also appears in yellow in this picture).

The tagging decision is based on one or more measured features. Each feature can contribute to the final decision, or not. If yes, the feature can contribute with a different weight.

A scoring mechanism can be based on one or more of the following: each feature has a contributing weight factor that is multiplied by the calculated feature score. Usually, all calculated feature scores are 0 to 1 normalized. The overall score is achieved by summing of each several contributing factors. Each factor is the multiplication of the calculated feature score or function of several calculated features scores, by its contributing weight factor. The overall score is then compared to Threshold or to other class scores. See Eq.'s 1-4 below.

Equations 1-4: Score of class x is sum of multiplication of normalized features values with weights. Classification decision is against Threshold or other score.

$$\begin{matrix}{Score}_{x} = {\sum }_{i = 1}^{N} {w}_{i} * {f}_{i} & Eq . 1 \\ {Score}_{x} = {\sum }_{i = 1}^{N} {w}_{i } * F ( {f}_{i} , {f}_{j} ) & Eq . 2 \\ {Score}_{i} > {Score}_{j} & Eq . 3 \\ {Score}_{i} > T & Eq . 4\end{matrix}$$

The classification (also referred to as tagging process) can be based on “soft decision” or “hard decision” methods.

In “hard decision” the output of each step is based on features values calculated for each label. The outcomes are compared against each other in order to draw a specific classification/tagging recommendation for each label derived from this step. Typically, this recommendation step has a binary 0-1 values only (correspond to no-yes).

In a soft decision, unlike hard decisions, the classification/tagging per label, is performed with a certain probability only. That is, we follow the same features calculations but the decision on classification/tagging takes into account several recommendations when each feature recommendation value can range from 0 to 1 and includes a ay number within.

For example, tagging an object as Skin with 70% probability means that the object is skin but with 70% certainty only. This certainty value does not exist for “hard-decision” making.

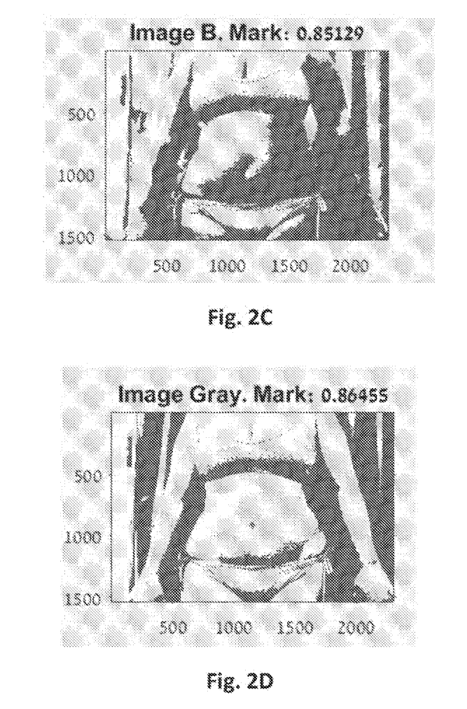

In some embodiments, the colors variance calculation is performed by a fast method, using the following steps:
1. Using randomly selected one class regions (one class means one color), and computing the color variance for each of those regions. See FIGS. 5A-5B for such regions showed as rectangles. FIGS. 5A, 5B show randomly selected rectangle areas from different areas of the image. These rectangle are used in order to assign a tagging (skin/non skin) for the different objects/classes.
2. Sampling each of the regions one or more times in order to cover all classes (colors) and in a predetermined quantity.
3. Calculating for each one-label regions a variance. One label means one class, also referred to as one color.
4. Calculating the average of the variances for every label.
5. Classifying one-label regions with highest average as skin. Alternatively, assigning each object with a foreground probability and a background probability. The foreground probability is the probability that the object is a foreground object (skin). The background probability is the probability that the object is a background object.

In another embodiment, colors variance calculation is performed on all pixels, first computing the color variance for each object and then performing steps 4, 5 above.

In another embodiment, classifying is based on area count. Either the biggest label count is classified as a foreground object (skin) or each object is assigned a foreground probability and a background probability.

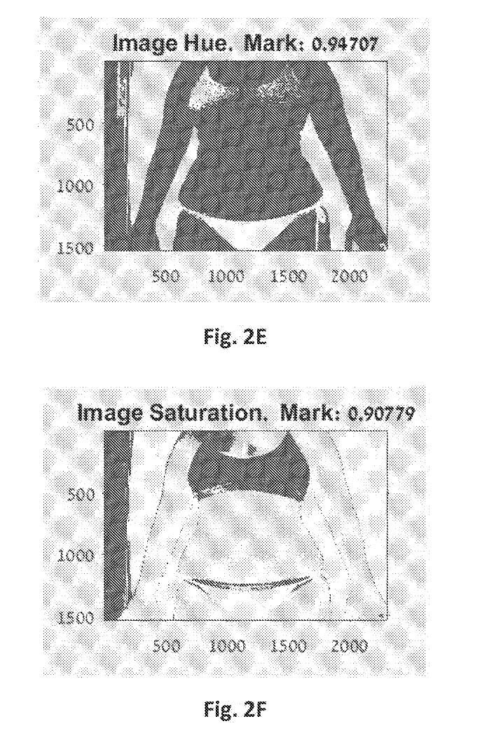

The biggest detected label is likely to be skin (foreground object). In FIG. 6B one can see the skin detection marked in deep blue, underwear in yellow while the background is marked in light blue. The original RGB image can be seen in FIG. 6A.

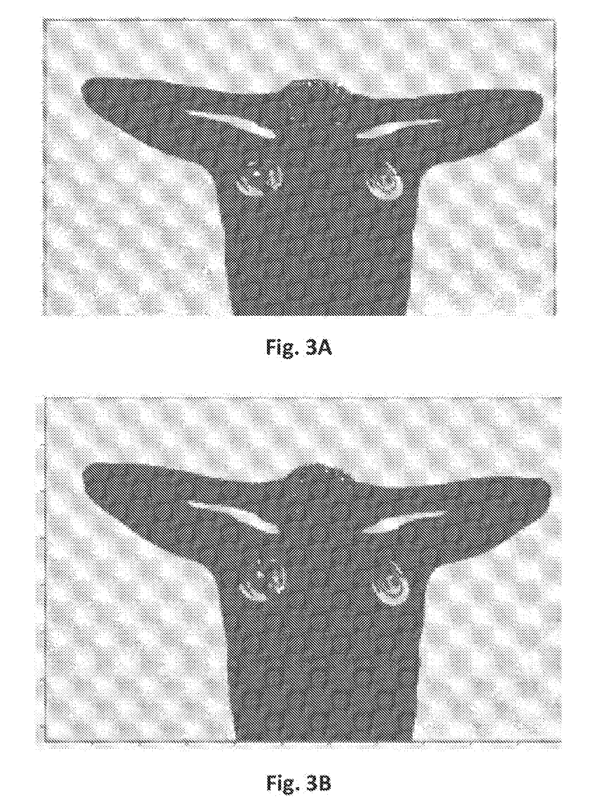

In some embodiments, classifying is based on central line(s) counting, comprising the steps of:
(i) counting the number of pixels per object along a plurality of central lines from top to down running along the center of the image as can bee seen in the black line marked in the center of FIG. 7.
(ii) classifying biggest pixel count objects as foreground objects. Alternatively, assigning foreground and background probabilities to each object. The foreground probability is the probability that the object is a foreground object (skin). The background probability is the probability that the object is a background object.
(iii) counting the number of labeling changes along a plurality of central lines; and
(iv) classifying smallest pixel count objects as underwear objects. Alternatively, assigning foreground and underwear probabilities per object.

In another embodiment, classifying is based on peripheral region counting, comprising the steps of:
(i) counting the number of pixels per object along a plurality of left and right side lines as can be seen in the black lines marked in the left & right sides of FIG. 7 and
(ii) classifying biggest pixel count objects as background objects. Alternatively, assigning foreground and background probabilities per object.

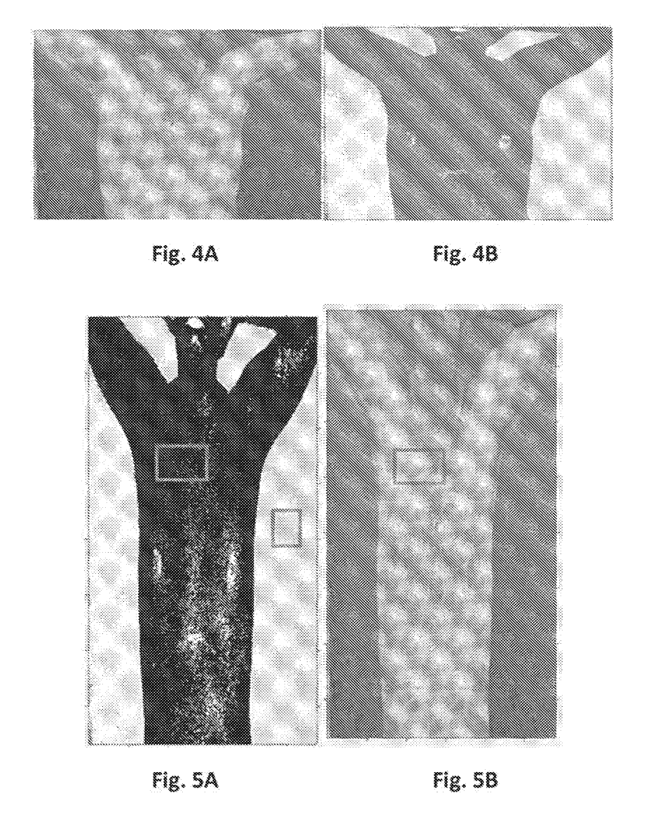

In some embodiments, classifying is based on objects color and proximity (resemblance) to skin color, comprising the steps of:
(i) defining a common color per object, this is the common value for the density estimation range that define the class, as shown in FIG. 8. For example, class 2 is defined between intensities range of 0.35-0.73 (values founded during the segmentation process). Its common value is 0.58 (highest count). In FIG. 8 the black lines are drawn in order to emphasis the classes boundaries.
(ii) skin color can have hue color of around 0.75-1,0-0.2, this can be seen in the marked star in FIG. 8 and
(iii) calculating the distance of each class to the defined skin color, and
(iv) classifying closest color objects as foreground objects or assign foreground and background probabilities per object.

In some embodiments, classifying is based on a scoring mechanism.

For example, the scoring mechanism can be based on assigning each feature of an object a contributing weight factor that is multiplied by the calculated feature score. Objects with the biggest mark are then classified as foreground objects, objects with smallest marks are classified as background objects and objects with the highest object transitions count are classified as underwear objects.

Classification (tagging) of objects can be done in several manners, for example:
Highest Mark is classified as Foreground (Skin);
Lowest Mark is classified as Background;
Highest objects Transitions count is classified as Underwear,
Smaller objects Transitions count is classified as tattoos/jewelry.

In some embodiments, the image orientation (portrait or landscape) is identified.

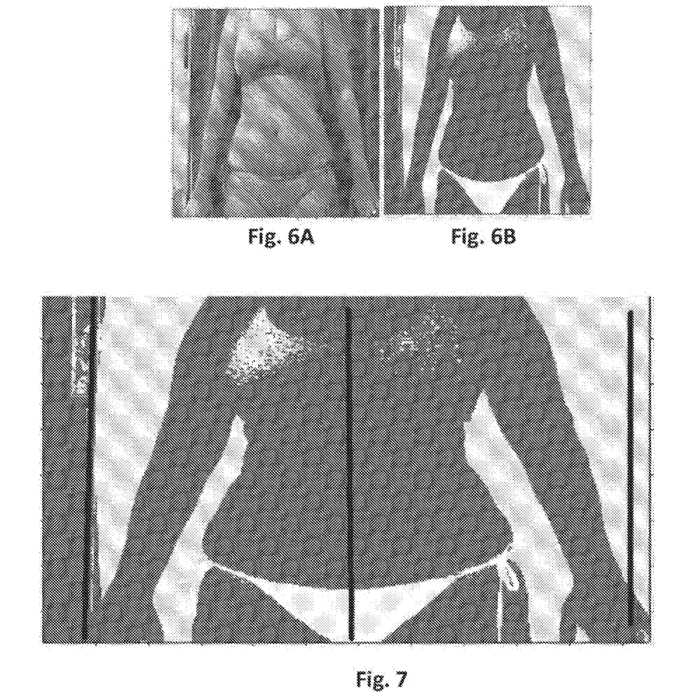

In some embodiments, image orientation is detected according to the following steps:
(i) detecting central lines as lines traversing from up to down and from left to right passing through the center of image or foreground center of mass; (ii) counting the number of foreground pixels along a plurality of central lines from top to down running along the center of the image as can be seen in the black line marked in the center of FIGS. 9A-9B.
(iii) counting the number of foreground and background pixels transitions along a plurality of central lines; and
(iv) determining the image as in portrait mode if count and transitions are higher and determining the image as in landscape mode if count and transitions are smaller. FIG. 9A is an example of the detection lines that count either from top to down or from left to right (FIG. 9B).

Alternatively, steps (i)-(iii) above are repeated with off-central lines and adding their scores to the mechanism of step (iv).

Alternatively, step (iv) can be done by applying Equations 2-4.

In some embodiments, jewelry/tattoos are detected. The detection is performed according to the following steps:
(i) Objects with non-skin tagging.
(ii) less then highest object transitions counts
(iii) very close (pixel count) to the skin tagging. In some embodiments, head hair is detected. Head hair detection is performed according to the following steps:
(i) scanning foreground pixels along top half central part;
(ii) counting 3D full color space density values of said foreground pixels;
(iii) clustering said values to a plurality of groups;
(iv) determining how close each cluster is to hair color;
(v) applying one or more edge filters on saturation or other color fields;
(vi) calculating variance of Edge filter;
determining based on steps (i) to (vi) if a cluster is hair or not, wherein if a cluster is classified as hair, approximating head shape as geometric circle/oval shape using a voting method and classifying said cluster as non-bald head part and excluding said cluster from Foreground/skin classification.

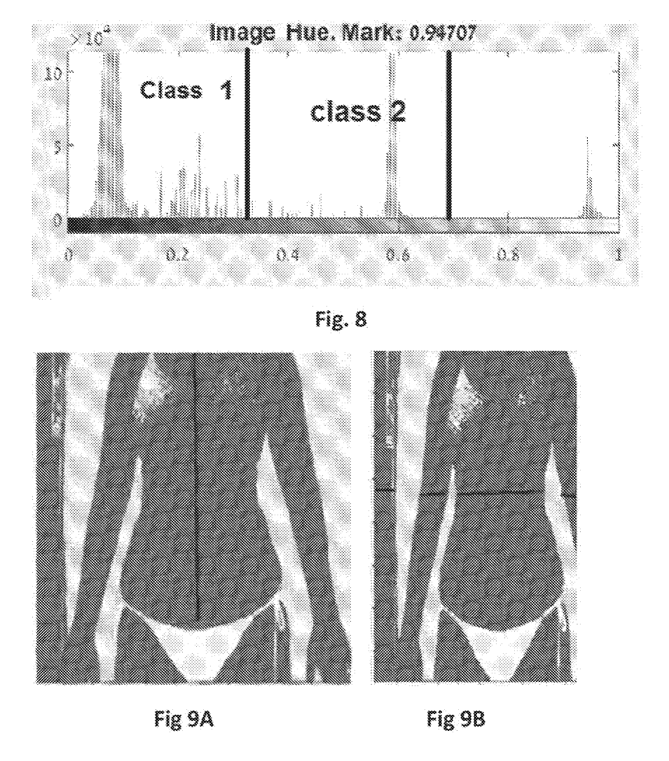

FIG. 10 is an example of applying edge filters. One can see strong edge response in the region of the head, these correspond to hairy patches pixels.

Although the invention has been described in detail, nevertheless changes and modifications, which do not depart from the teachings of the present invention, will be evident to those skilled in the art. Such changes and modifications are deemed to come within the purview of the present invention and the appended claims.

It will be readily apparent that the various methods and algorithms described herein may be implemented by, e.g., appropriately programmed general purpose computers and computing devices. Typically a processor (e.g., one or more microprocessors) will receive instructions from a memory or like device, and execute those instructions, thereby performing one or more processes defined by those instructions. Further, programs that implement such methods and algorithms may be stored and transmitted using a variety of media in a number of manners. In some embodiments, hard-wired circuitry or custom hardware may be used in place of, or in combination with, software instructions for implementation of the processes of various embodiments. Thus, embodiments are not limited to any specific combination of hardware and software.

A “processor” means any one or more microprocessors, central processing units (CPUs), computing devices, microcontrollers, digital signal processors, or like devices.

The term “computer-readable medium” refers to any medium that participates in providing data (e.g., instructions) which may b read by a computer, a processor or a like device. Such a medium may take many forms, including but not limited to, non-volatile media, volatile media, and transmission media. Non-volatile media include, for example, optical or magnetic disk and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and Infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements or stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviors of a database can be used to implement various processes, such as the described herein. In addition, the databases may, in a known manner, be stored locally or remotely from a device which accesses data in such a database.

The present invention be configured to work in a network environment including a computer that is in communication, via a communications network, with one or more devices. The computer may communicate with the devices directly or indirectly, via a wired or wireless medium such as the Internet, LAN, WAN or Ethernet, Token Ring, or via any appropriate communications means or combination of communications means. Each of the devices may comprise computers, such as those based on the Intel®, Pentium®, or Centrino™, processor, that are adapted to communicate with the computer. Any number and type of machines may be in communication with the computer.

1. A computing system comprising:
at least one processor; and
at least one memory communicatively coupled to the at least one processor comprising computer-readable instructions that when executed by the at least one processor cause the computing system to implement a method for analyzing a digital photograph and identifying skin areas, the method comprising the steps of:
(i) transforming said digital photograph to a plurality of color bases candidates, and choosing one candidate based on highest score plane;
(ii) performing a segmentation of said digital photograph to a plurality of objects based on maximal separations between different object; and
(iii) classifying said objects to one or more of the following categories: skin, background or underwear, wherein said classification is based on chosen plane calculation, comprising the steps of:
a) using one or more selected label regions, computing a statistical decisor for each area;
b) sampling each of said regions one or more times;
c) calculating for each one-label region the statistical decisor;
d) calculating the statistical decisor for one or more features of every label; and
e) classifying one-label regions with matching statistical decisor value as skin or assigning each object with a foreground probability and a background probability.

2. The computing system according to claim 1, wherein said color base is hue-saturation-lightness (HSL), hue-saturation-value (HSV), hue-saturation-intensity (HSI) or La*b* (CIELa*b*) or RGB.

3. The computing system according to claim 1, wherein said segmentation is based on hue or saturation or lightness or any one of the “regular” R or G or B planes.

4. The computing system according to claim 1, wherein said segmentation comprises the following steps:
(i) detecting thresholds using density estimation and maximization of inter class variation; and
(ii) smoothing and unifying objects in said digital photographs based on filters and proximity.

5. The computing system according to claim 1, wherein the statistical decisor comprises average, common, median, mode, variance, standard deviation, (minimum+maximum)/2, any mathematical moment or any combination thereof.

6. The computing system according to claim 1, wherein said feature comprises gray intensity, red/green/blue values, saturation value or any combination thereof.

7. The computing system according to claim 1, wherein steps (a) to (e) of the classification are substituted by computing the statistical decisor for the entire object.

8. The computing system according to claim 1, wherein said classifying is based on object size count where either the biggest pixel count is classified as a foreground object or assigning each object with a foreground probability and a background probability.

9. The computing system according to claim 1, wherein said classifying is based on central region counting, comprising the steps of:
(i) counting the number of pixels per object along a plurality of central lines;
(ii) classifying biggest pixel count objects as foreground objects or assign foreground and background probabilities per object;
(iii) counting the number of objects transitions along a plurality of central lines; and
(iv) classifying smallest pixel count objects as underwear objects or assign foreground and underwear probabilities per object.

10. The computing system according to claim 1, wherein said classifying is based on peripheral region counting, comprising the steps of:
(i) counting the number of pixels per object along a plurality of peripheral left and right side lines; and
(ii) classifying biggest pixel count objects as background objects or assign foreground and background probabilities per object.

11. The computing system according to claim 1, wherein said classifying is based on objects color and proximity to skin color, comprising the steps of:
(i) defining a common color per object; and
(ii) classifying closest color objects as foreground objects or assign foreground and underwear probabilities per object.

12. The computing system according to claim 1, wherein said classifying is based on a scoring mechanism.

13. The computing system according to claim 12, wherein said scoring mechanism is based on assigning each feature of an object a contributing weight factor that is multiplied by the calculated feature score, wherein the pixels labels with the biggest mark are classified as foreground objects, objects with smallest marks are classified as background objects, and objects with the highest object transitions count are classified as underwear objects.

14. The computing system according to claim 1, further comprising the step of determining image orientation.

15. The computing system according to claim 14, wherein said image orientation is detected according to the following steps:
(i) detecting central lines as lines traversing from up to down and from left to right passing through the center of image or foreground center of mass;
(ii) counting the number of foreground pixels along a plurality of central lines;
(iii) counting the number of foreground and background pixels transitions along a plurality of central lines; and
(iv) determining the image as in portrait mode if count and transitions are higher and determining the image as in landscape mode if count and transitions are smaller.

16. The computing system according to claim 15, wherein steps (i)-(iii) are repeated with off-central lines and adding their scores to the mechanism of step (iv).

17. The computing system according to claim 1, further comprising the step of head hair detection.

18. The computing system according to claim 17, wherein said head hair detection is performed according to the following steps:
(i) scanning foreground pixels along top half central part;
(ii) counting 3D full color space density values of said foreground pixels;
(iii) clustering said values to a plurality of groups;
(iv) determining how close each cluster is to hair color;
(v) applying one or more edge filters on saturation or other color fields;
(vi) calculating variance of edge filter;
(vii) determining based on steps (i) to (vi) if a given cluster is hair or not, wherein if a cluster is classified as hair, approximating head shape as geometric circle/oval shape using a voting method and classifying said cluster as non-bald head part and excluding said cluster from foreground/skin classification.

19. The computing system according to claim 1, wherein digital photograph was taken according to a total body photography protocol.

20. (canceled)

21. A computerized system comprising a processor and a memory communicatively coupled to the processor comprising computer-readable instructions for analyzing a digital photograph and identifying skin areas, the system comprising:
(i) a software transformation module adapted for transforming said digital photograph to a plurality of color bases candidates, and choosing one candidate based on highest score plane;
(ii) a software segmentation module adapted for performing a segmentation of said digital photograph to a plurality of objects based on maximal separations between different object; and
(iii) a software classification module adapted for classifying said objects to one or more of the following categories: skin, background or underwear, wherein said classification is based on chosen plane calculation, comprising the steps of:
a) using one or more selected label regions, computing a statistical decisor for each area;
b) sampling each of said regions one or more times;
c) calculating for each one-label region the statistical decisor;
d) calculating the statistical decisor for one or more features of every label; and
e) classifying one-label regions with matching statistical decisor value as skin or assigning each object with a foreground probability and a background probability.

22-39 (canceled)
